(19) United States
(12) Patent Application Publication
(10) Pub. No.: US 2025/0268791 A1
(43) Pub. Date: Aug. 28, 2025

(54) Apparatus and System for Remote Medication-Adherence Monitoring

(71) Applicant: Tenovi, Portsmouth, NH (US)

(72) Inventors: Nizan Friedman, Irvine, CA (US); Daniel K. Zondervan, Irvine, CA (US)

(21) Appl. No.: 19/064,283

(22) Filed: Feb. 26, 2025

Related U.S. Application Data
(60) Provisional application No. 63/558,075, filed on Feb. 26, 2024.

(51) Int. Cl. A61J 1/03; A61J 7/04; G16H 20/13

(52) U.S. Cl. CPC A61J 1/035; A61J 7/0418; G16H 20/13

(57) ABSTRACT

In one embodiment, a smart pill container of a system for remote medication-adherence monitoring includes multiple pill compartments each defining an interior space in which one or more pills can be stored, multiple pill compartment closures, one pill compartment closure being associated with each pill compartment and being configured to close its associated pill compartment, each pill compartment closure including indicia that at least identifies a day of the week on which the one or more pills contained within the associated pill compartment are to be ingested by a patient, multiple pill compartment sensors, one pill compartment sensor being associated with each pill compartment and being configured to detect opening of the pill compartment, a clock configured to track the current day and time, and a microcontroller configured to log data when opening of a pill compartment is detected.

### CROSS-REFERENCE TO RELATED APPLICATION

This application claims priority to co-pending U.S. Provisional Application Ser. No. 63/558,075, filed Feb. 26, 2024, which is hereby incorporated by reference herein in its entirety.

### BACKGROUND

Approximately 117 million Americans live with one or more chronic diseases, which often require multiple lifelong medications to control. Consistent adherence to a medication regimen is an important factor in the management of disease progression. However, it is estimated that nearly 50% of patients do not take their medications as prescribed, resulting in increased morbidity, mortality, and healthcare costs. The burden of following a medication plan can be overwhelming, particularly for individuals managing multiple medications. Patients often forget to take their medication on time, leading to errors, especially when regimens are more complex. Nonadherence can also be caused by health literacy limitations, mistrust in healthcare providers, and fear of side effects. These challenges highlight the need for innovative approaches to medication adherence management.

One promising approach is the use of remote therapeutic monitoring programs to (1) monitor the times at which patients take their pills and (2) facilitate regular, data-driven communication with a care provider to reinforce accountability, address barriers, monitor for side effects, and educate patients. The U.S. healthcare system has recently embraced this remote monitoring paradigm after both the Covid-19 public health emergency and several pivotal studies demonstrating the efficacy of remote monitoring for improving difficult-to-change health outcomes. Medicare created new reimbursement codes for remote patient monitoring (RPM) in 2020 and remote therapeutic monitoring (RTM) in 2022. The RTM codes enable healthcare providers to remotely monitor non-physiological data (i.e., medication adherence) and remotely interact with patients for up to 40 minutes per month, creating a novel opportunity to address medication adherence via RTM in an economically viable and proactive manner.

Given this new support for remote monitoring of medication adherence, it can be appreciated that it would be desirable to have effective apparatuses and systems for remote medication-adherence monitoring.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a perspective view of an embodiment of a smart pill container configured to enable remote medication-adherence monitoring.

FIG. 2 is a further perspective view of the smart pill container of FIG. 1, shown with a pill compartment of the container in an open orientation.

FIG. 3 is an exploded perspective view of the smart pill container of FIG. 1, shown with a top cover and a first pill compartment module removed from the remainder of the container.

FIG. 4 is a detail perspective view of the interior of a base of the smart pill container of FIG. 1 that shows pill compartment sensors mounted to an internal circuit board of the container that are associated with the pill compartments of the first pill compartment module shown in FIG. 3.

FIG. 5 is a detail perspective view of the interior of the base of the smart pill container of FIG. 1 that shows various internal electrical components of the container mounted to the internal circuit board.

FIG. 6 is a schematic view of an embodiment of a system for remote medication-adherence monitoring, the system including the smart pill container shown in FIGS. 1-5.

### DETAILED DESCRIPTION

As described above, it would be desirable to have an effective apparatus and system for remote medication-adherence monitoring. Example embodiments of such an apparatus and system are disclosed herein.

In some embodiments, a system for remote medication-adherence monitoring comprises a smart pill container that includes multiple pill compartments associated with particular days of the week and, optionally, with particular times of the days. Each pill compartment includes a closure, such as a lid, that can be opened by a user (e.g., patient or local caregiver) to access the interior of the compartment for the purpose of adding pills to or removing pills from the container. Associated with each pill compartment is a pill compartment sensor that is configured to detect when the closure of a compartment is opened. When a pill compartment is accessed by opening its closure, a pill compartment open event is logged by both locally storing data that identifies the particular compartment that was accessed as well as the day and time it was accessed, and wirelessly transmitting the data to a HIPAA-compliant server.

In some embodiments, the pill compartment open event data, and optionally other data described below, is wirelessly transmitted from the smart pill container to a nearby gateway device using a short-range wireless communication standard, such as Bluetooth™, and the gateway device transmits the data to the HIPAA-compliant server using a cellular network. In some embodiments, the smart pill container can be placed in a refill mode, for example, by pressing a refill button on the container, that temporarily suspends the logging of pill compartment open events to enable a user to refill the pill compartments after they have been depleted, for instance, at the end of each week.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

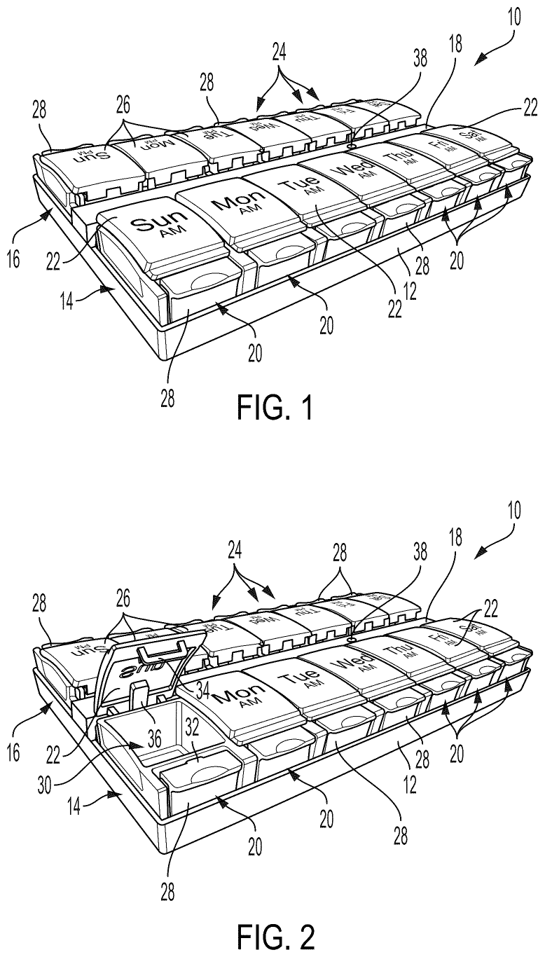

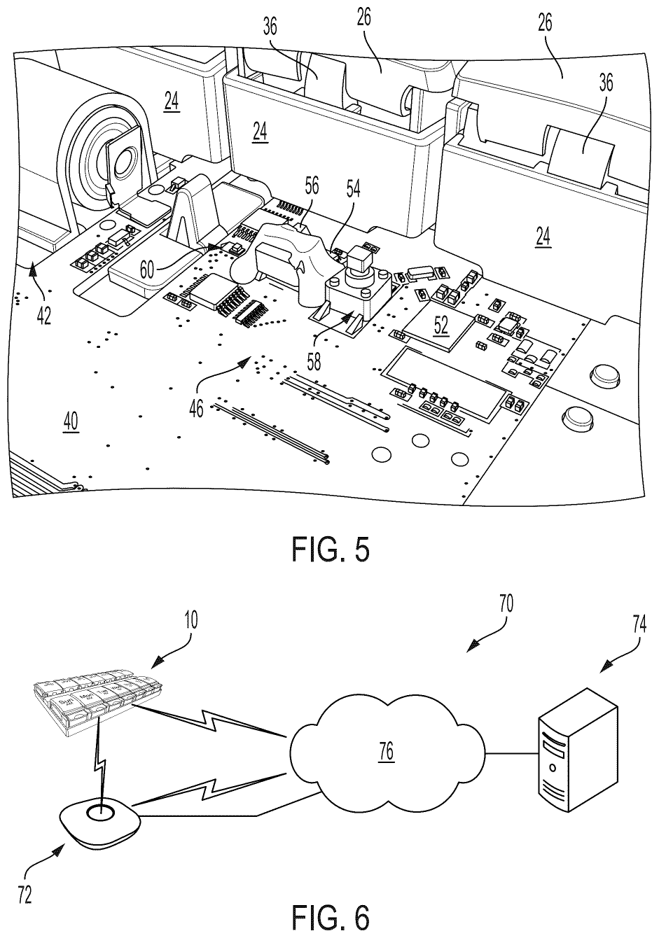

FIG. 1 illustrates an embodiment of a smart pill container 10 that is configured to facilitate remote medication-adherence monitoring. As shown in the figure, the container 10 generally comprises a base 12 that supports and to which are mounted one or more pill compartment modules. In the example of FIG. 1, the container 10 includes two such modules: a first pill compartment module 14 and a second pill compartment module 16. Positioned between the modules 14, 16 is a top cover 18 that encapsulates internal electrical components of the smart pill container 10, which are described below in relation to FIG. 5. In the illustrated embodiment, the first pill compartment module 14 includes seven separate pill compartments 20, each having a closure in the form of a lid 22 that has a label that identifies a day of the week as well as a time of day. From left to right in the orientation of FIG. 1, the lids 22 of the first pill compartment module 14 are labeled “Sun,” “Mon,” “Tue,” “Wed,” “Thu,” “Fri,” and “Sat.” In addition, each lid includes an “AM” designation. As with standard pill organizers, that indicia provides an indication to the user as to when the one or more pills contained within the associated pill compartment 20 are to be taken (i.e., ingested) by the patient. Accordingly, pills contained in the left-most compartment 20 of the first pill compartment module 14 are to be taken on Sunday in the morning, the pills contained in the next compartment to the right are to be taken on Monday in the morning, and so forth. Because each of the compartments 20 is intended to contain pills that are to be taken in the morning of the indicated day of the week, the first pill compartment module 14 may be referred to as the AM or morning pill compartment module.

The second pill compartment module 16 has a configuration similar to that of the first pill compartment module 14. In particular, the second pill compartment module 16 also includes seven separate pill compartments 24, each having a closure in the form of a lid 26 that has a label that identifies a day of the week as well as a time of day. When the smart pill container is rotated 180 degrees from its orientation shown in FIG. 1 so that the second pill compartment module 16 faces the user, the lids 26 are labeled from left to right as “Sun,” “Mon,” “Tue,” “Wed,” “Thu,” “Fri,” and “Sat.” Instead of further including an “AM” designation, however, the lids 26 of the second pill compartment module 16 include a “PM” designation. Accordingly, pills contained in the left-most compartment 24 of the second pill compartment module 16 are to be taken on Sunday in the evening, the pills contained in the next compartment to the right are to be taken Monday in the evening, and so forth. Because each of the compartments 24 is intended to contain pills that are to be taken in the evening of the indicated day of the week, the second pill compartment module 16 may be referred to as the PM or evening module.

Associated with each pill compartment 20, 24 are closure release buttons 28. As is apparent from FIG. 1, one release button 28 is provided for each pill compartment 20, 24. In the illustrated embodiment, the release buttons 28 are located near the outer edge of each lid 22, 26. When a release button 28 associated with a lid 22 or 26 is activated by the user, for example, by pressing the button downward, the lid is released and opens to provide access to the interior of the pill compartment 20 or 24 that the lid covers. An example of this is illustrated in FIG. 2, which shows the “Sun AM” lid in an open position such that the interior space 30 defined by the “Sun AM” pill compartment is accessible. In some embodiments, the release button 28 comprises a small tab 32 at an upper portion of the button that engages a latch 34 provided at the outer edge of the lid 22 when the lid is in the closed position shown in FIG. 1.

When the release button 28 is pressed downward, the upper portion of the button pivots outward away from the lid 22 so that the tab 32 disengages from the latch 34. When the latch 34 is so disengaged, the lid 22 can be pivoted upward away from the release button 28 to provide access to the interior space 30 of the compartment 20. In some embodiments, a spring element 36 is provided for each pill compartment 20, 24 that applies an upward (opening) force to the lid 22 that causes the lid to spring open once its latch 34 has been disengaged from its associated release button 28. Notably, each lid 22, 26 of each pill compartment 20, 24 can operate in a similar manner to provide on-demand access to the interior of any compartment and, therefore, any pills it may contain.

With reference back to FIG. 1, a refill button 38 is integrated into the top cover 18. As is described below, when the refill button 38 is pressed, a user can open each of the pill compartments 20, 24 without individual pill compartment open events being logged for those compartments.

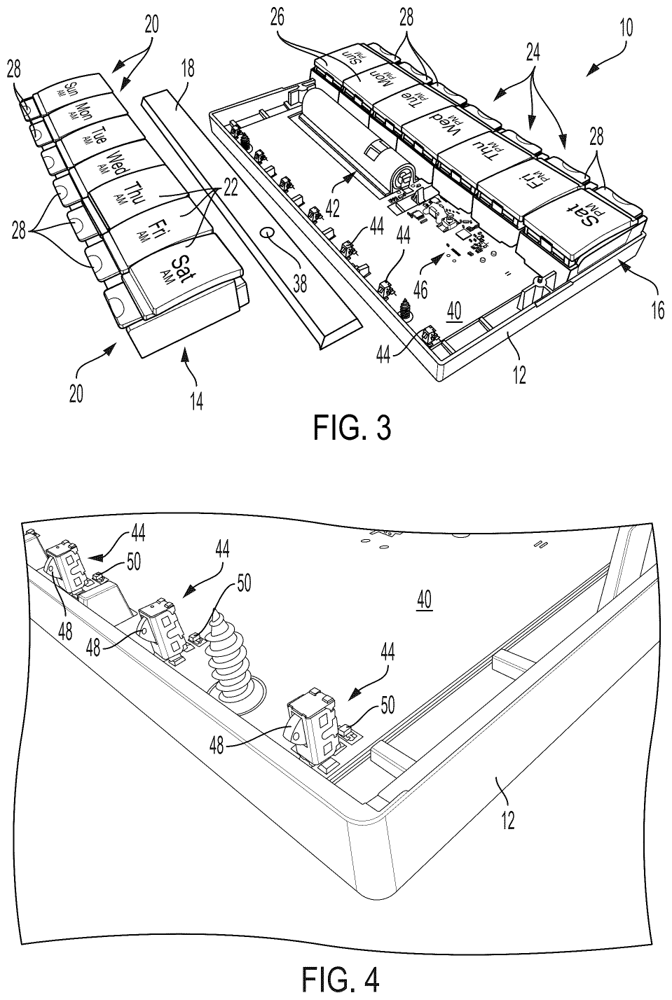

FIG. 3 shows the smart pill container 10 with the first pill compartment module 14 and the top cover 18 removed from the remainder of the container. With those components removed, the interior of the tray-shaped base 12 is visible, as is a printed circuit board 40 that is provided within the base beneath the first and second pill compartment modules 14, 16. Mounted to the circuit board 40 is a battery compartment 42, which is configured to receive one or more batteries that are used to power the smart pill container 10. Also mounted to the circuit board 40 are pill compartment sensors 44, which are configured to detect opening of the pill compartments 20, 24, and various other electrical components 46, which are used to log and transmit data relating to pill compartment open events.

The pill compartment sensors 44 are shown more clearly in the detail view of FIG. 4. There is one sensor 44 associated with each pill compartment 20, 24, and each sensor extends upward from the circuit board 40 next to a release button 28 associated with the pill compartment. In the illustrated embodiment, each sensor 44 comprises an electrical switch having a spring-loaded trigger 48 that extends outward from the switch in a direction facing an inner surface of its associated release button 28. In this example, the trigger 48 of each sensor 44 is immediately adjacent to an inner surface of a lower portion of the release button 28. In such a case, when the release button 28 is pressed downward by the user to pivot the upper portion of the button away from its associated lid 22, 26, the lower portion of the release button is pivoted into contact with the switch's trigger 48, which trips (i.e., activates) the switch and enables detection of opening of the associated pill compartment. Operating in that manner, the smart pill container 10 can detect whenever a particular pill compartment has been opened and, therefore, infer when the patient is taking the pills contained therein.

Also mounted to the circuit board 40 are lights 50, in the form of light emitting diodes (LEDs), which are individually associated with the various pill compartments 20, 24. As with the sensors 44, one light 50 is provided for each of the pill compartments 20, 24. As described below, the lights 50 are used to provide an indication as to which pills (i.e., the pills of which pill compartment) are to be taken by the patient whenever one or more of the pill compartments 20, 24 is opened, except for when the smart pill container 10 is operating in a refill mode.

Referring next to FIG. 5, illustrated are various electrical components mounted to the circuit board 40 that are used to operate the smart pill container 10. Those components include a microcontroller 52, a real-time clock 54 and its crystal oscillator 56, and a refill switch 58. The microcontroller 52 controls the operation of the other electrical components of the smart pill container 10 and includes both a wireless (e.g., Bluetooth™) transceiver module and local solid-state (e.g., flash) storage memory. The clock 54 and its oscillator 56 are used to keep track of the current day and time when the smart pill container 10 is in use so that the current day/time can be referenced any time an event is to be logged. The refill switch 58 is associated with the refill button 38 integrated into the top cover 18 and is tripped any time that button is pressed by a user. Also visible in FIG. 5 is a further light 60 (e.g., LED) that can be used to convey the connection status of the smart pill container 10 to another device, such as a gateway device described below in relation to FIG. 6.

The configuration of the smart pill container 10 having been discussed above, operation of the container in facilitating remote medication-adherence monitoring will now be discussed with reference to FIG. 6, which illustrates an embodiment of a system 70 for remote medication-adherence monitoring. As shown in FIG. 6, the system 70 includes the smart pill container 10 as well as a local gateway device 72 that can be used with the container. To initiate use of the system 70, the gateway device 72 can be plugged into a power outlet and batteries can be inserted into the battery compartment 42 of the smart pill container 10 so as to power both devices.

Once both the gateway device 72 and smart pill container 10 have powered up, a wireless connection can be established to enable bi-directional communication between the two devices. Like the smart pill container 10, the gateway device 72 can include a wireless transceiver module configured to transmit and receive data using a short-range wireless communication standard (e.g., Bluetooth™). In some embodiments, a pairing button (not shown) on the smart pill container 10 can be pressed to place the container in a pairing mode. When pairing has been initiated, the light 60 provided on the circuit board 40 of the smart pill container 10 will flash while the container attempts to connect to the gateway device 70. After a connection has been established between the smart pill container 10 and the gateway device 70, the light 60 will shine continuously to communicate proper connection a user, such as the patient or local caregiver. In addition to establishing a connection with the smart pill container 10, the gateway device 70 can establish a connection with a remote HIPAA-compliant server 74 via a network 76. In some embodiments, the network 76 comprises a cellular network and the gateway device 70 communicates with the server 74 via cellular communications. In other embodiments, the gateway device 70 can have a wired internet connection and can communicate with the server 74 via that connection. Irrespective of the particular nature of the connection established between the gateway device 70 and the server 74, the gateway device can obtain the current day and time from the server.

Once a connection has been established between the smart pill container 10 and the gateway device 70, the gateway device updates the smart pill container with the current day and time, which the smart pill container can then track on its own going forward using its real-time clock 54. Before the smart pill container 10 can be used to monitor the patient's medication adherence, the container must be filled with the medication (i.e., pills) that the patient is to take. To avoid a situation in which the opening of multiple pill compartments 20, 24 are incorrectly interpreted as the patient taking all of the medications stored in those compartments, the smart pill container 10 can be placed in a refill mode prior to filling. The user can do that by pressing the refill button 38 integrated into the top cover 18 of the smart pill container 10 prior to opening any of the pill compartments. When that button 38 is pressed a first time, the refill switch 58 is tripped and the smart pill container 10 enters the refill mode. During the refill mode, the smart pill container 10 will not interpret the opening of any of the pill compartments as pill compartment open events indicative of medication being taken. In some embodiments, each of the lights 50 associated with the pill compartments can flash to communicate to the user that the smart pill container 10 is operating in refill mode. Regardless, once refill mode has been entered, the user can fill the pill compartments with the various medications that the patient is to take on the days and times identified on the lids 22, 26 of the compartments.

After each of the pill compartments has been filled with the appropriate medications in accordance with the patient's medication schedule, the refill mode can be exited and normal operation can be initiated by pressing the refill button 38 a second time. When the user does that, the lights 50 will stop flashing and the smart pill box 10 is prepared for normal use.

From this point, the smart pill container 10 can be used like a conventional pill organizer with the user opening a compartment on the day and at the time designated on the lid of the compartment, and the patient taking the pills contained therein. For example, if it is Tuesday morning and the patient has not yet taken any medication stored in the smart pill container 10, the user would open the “Tue AM” compartment and the patient would take the pills contained therein.

Whenever a pill compartment is opened, the pill compartment sensor 44 associated with the compartment detects opening of the compartment. More specifically, when a user presses the release button 28 associated with a given pill compartment to enable the lid of the compartment to open, the button 48 that extends outward from the switch of the sensor 44 is pressed inward by the release button and the switch is triggered, thereby registering a pill compartment open event. As each sensor 44 is associated with one particular pill compartment and as the smart pill container 10 tracks the current day and time, the container is aware as to which pill compartment should be opened at any particular time. To provide an indication, (e.g., notification or confirmation) to the user as to the correct pill compartment from which to take pills on that particular day and at that particular time, the light 50 associated with the correct pill compartment illuminates when any pill compartment is opened. In other words, the opening of any pill compartment wakes the smart pill container 10 and causes it to illuminate the pill compartment that contains the pills that should be taken by the user on that day and at that time, regardless of which pill compartment has been opened by the user.

As an operation example, if it is Wednesday morning and the user opens the “Wed AM” pill compartment, the “Wed AM” pill compartment will illuminate, thereby providing confirmation to the user that he or she has accessed the correct pills. If, however, it is Wednesday morning and the user mistakenly opens the “Wed PM” pill compartment, the “Wed AM” pill compartment will still illuminate (and the “Wed PM” pill compartment will not illuminate), thereby notifying the user that he or she has accessed the wrong pills. The user can then close the “Wed PM” pill compartment and open the illuminated “Wed AM” pill compartment to correct the error before the patient ingests the wrong pills for that particular time of day.

Any time a pill compartment is opened, whether it is the correct pill compartment or an incorrect pill compartment for the current day and time, the smart pill container 10 logs the opening as a pill compartment open event by storing open event data associated with that opening. The open event data includes an identification of the particular pill compartment that was opened as well as an identification of the day and time the compartment was opened. That data, through inference, provides an indication as to what medications were taken by the patient and when. Optionally, incorrect pill compartment open events can be flagged as such to provide an indication as to when a user opens the wrong pill compartment.

As noted above, the open event data can be locally stored on the smart pill container 10, such as within the storage memory of the microprocessor 52. In addition, the data can be wirelessly transmitted to the gateway device 72 using the short-range wireless communication standard whenever the gateway device is in communication range and has a wireless connection to the smart pill container 10. The open event data received by the gateway device 72 can then be transmitted to the HIPAA-compliant server 74, for example, across a cellular network. In some embodiments, the open event data can then be shared with third parties, such as remote monitoring services, that can store, analyze, and share the data with relevant parties. For instance, such a service may aggregate the open event data of the patient and present the aggregated data to the patient's healthcare provider using a graphical representation accessible using a web-based interface. In such a case, a full picture of the patient's adherence to a medication regimen over given periods of time (e.g., one or more weeks, one or more months, etc.) can be assessed by the patient's healthcare provider, who can determine what actions, if any, should be taken in regard to the patient's healthcare. If, for example, the patient's adherence to the medication regimen is assessed to be inadequate, the healthcare provider may wish to communicate with the patient and provide suggestions intended to improve the patient's adherence to the regimen.

In situations in which the smart pill container 10 is no longer in communication range of the gateway device 72, for example, if the patient is out of town and left the gateway device at home, the container will store the pill compartment open events locally until such time the container is back in communication range of the gateway device and can download the stored data to the gateway device. As the smart pill container 10 has sufficient space to store numerous pill compartment open events, medication adherence data will be preserved even if the container is out of communication range of the gateway device 72 for multiple weeks. In situations in which the smart pill container 10 does not know the current day or time, the container will still log pill compartment open events along with an indication that the day and time information was not available when those events were logged. Once the smart pill container 10 is back in communication range of the gateway device 72, however, the container can obtain the correct day and time from the gateway device.

Notably, the smart pill container 10 can log events beyond pill compartment open events. For example, in some embodiments, the smart pill container 10 logs whenever the refill button 38 is pressed so that the days and times when the container is refilled with medication can also be tracked.

Various modifications can be made to the configuration and operation to the apparatuses and systems described above. For example, instead of or in addition to the correct pill compartment for the current day and time illuminating when a user opens one of the pill compartments, alternative actions detected by the smart pill container can cause such illumination. For example, if a motion sensor, such as an accelerometer, were added to the smart pill container, the correct pill compartment could be illuminated upon detection of the container being picked up or otherwise moved by the user. Alternatively, if the smart pill container included one or more external touch sensors, such as capacitive or resistive sensors, the correct pill compartment could be illuminated upon detection of the container being gripped or otherwise touched by the user. In such cases, some form of detected interaction between the user and the smart pill container can cause the container to light up the correct pill compartment as a way to communicate to the user which pills the patient should take next.

1. A smart pill container comprising:
multiple pill compartments each defining an interior space in which one or more pills can be stored;
multiple pill compartment closures, one pill compartment closure being associated with each pill compartment and being configured to close its associated pill compartment, each pill compartment closure including indicia that at least identifies a day of the week on which the one or more pills contained within the associated pill compartment are to be ingested by a patient;
multiple pill compartment sensors, one pill compartment sensor being associated with each pill compartment and being configured to detect opening of its associated pill compartment;
a clock configured to track the current day and time; and
a microcontroller configured to log data when opening of a pill compartment is detected.

2. The smart pill container of claim 1, wherein there is one pill compartment for each day of the week.

3. The smart pill container of claim 1, wherein there are two pill compartments for each day of the week, including a first pill compartment for a first time of day and a second pill compartment for a second time of day.

4. The smart pill container of claim 3, wherein the pill compartment closure for the first pill compartment includes indicia that identifies the morning and the pill compartment closure for the second pill compartment includes indicia that identifies the evening.

5. The smart pill container of claim 1, wherein each pill compartment closure comprises a lid that can be pivoted upward to open the associated pill compartment and provide access to any pills contained within the pill compartment.

6. The smart pill container of claim 5, further comprising multiple release buttons, one release button associated with each lid, wherein each lid pivots open when its associated release button is pressed by a user.

7. The smart pill container of claim 6, wherein pressing of a release button also trips the pill compartment sensor of the associated pill compartment.

8. The smart pill container of claim 7, wherein the pill compartment sensors each comprise an electrical switch and wherein the release button trips the pill compartment sensor by pressing a trigger of the switch.

9. The smart pill container of claim 1, wherein the microcontroller is configured to store on the smart pill container open event data when opening of a pill compartment is detected, wherein the open event data identifies which pill compartment was opened and on what day and at what time.

10. The smart pill container of claim 1, further comprising a wireless transceiver module configured to transmit and receive data using a short-range wireless communication standard.

11. The smart pill container of claim 10, wherein the microcontroller is configured to wirelessly transmit to a separate device the open event data when opening of the pill compartment is detected, wherein the open event data identifies which pill compartment was opened and on what day and at what time.

12. The smart pill container of claim 1, further comprising multiple lights, one light being associated with each pill compartment.

13. The smart pill container of claim 12, wherein the microcontroller is configured to illuminate the light associated with the pill compartment corresponding to the current day and time when opening of any of the pill compartments is detected.

14. The smart pill container of claim 1, further comprising a refill button, wherein the microcontroller is configured to pause logging of data pertaining to detection of opening of pill compartments in a refill mode that is initiated when the refill button has been pressed by a user.

15. A smart pill container comprising:
multiple pill compartments each defining an interior space in which one or more pills can be stored;
multiple pill compartment lids, one pill compartment lid being associated with each pill compartment and being configured to close its associated pill compartment, each pill compartment lid including indicia that identifies a day of the week and a time of day at which the one or more pills contained within the associated pill compartment are to be ingested by a patient;
multiple pill compartment sensors, one pill compartment sensor being associated with each pill compartment and being configured to detect opening of its associated pill compartment;
a clock configured to track the current day and time;
a wireless transceiver module configured to transmit and receive data using a short-range wireless communication standard; and
a microcontroller configured to log open event data when opening of a pill compartment is detected, wherein the open event data identifies which pill compartment was opened and on what day and at what time, the microcontroller further being configured to both store the open event data on the smart pill container and wirelessly transmit the open event data to another device.

16. A system for remote medication-adherence monitoring, the system comprising:
a smart pill container including multiple pill compartments each defining an interior space in which one or more pills can be stored, multiple pill compartment lids, one pill compartment lid being associated with each pill compartment and being configured to close its associated pill compartment, each pill compartment lid including indicia that at least identifies a day of the week on which the one or more pills contained within the associated pill compartment are to be ingested by a patient, multiple pill compartment sensors, one pill compartment sensor being associated with each pill compartment and being configured to detect opening of the pill compartment, a clock configured to track the current day and time, a wireless transceiver module configured to transmit and receive data using a short-range wireless communication standard, and a microcontroller configured to log open event data when opening of a pill compartment is detected; and
a gateway device configured to transmit and receive data using a short-range wireless communication standard and further configured to transmit and receive data via a cellular communication standard;
wherein the microcontroller is configured to wirelessly transmit logged open event data to the gateway device using the short-range wireless communication standard and wherein the gateway device is configured to wirelessly transmit the received open event data to another device using the cellular communication standard.

17. The system of claim 16, wherein the smart pill container comprises two pill compartments for each day of the week, including a first pill compartment for the morning and a second pill compartment for the evening.

18. The system of claim 16, wherein the smart pill container further comprises a pill compartment sensor associated with each pill compartment lid, each pill compartment sensor being configured to detect opening of its associated lid.

19. The system of claim 18, wherein the smart pill container further comprises a release button associated with each pill compartment lid, each release button being configured to open its associated lid and trip the pill compartment sensor associated with that lid when the release button is pressed by a user.

20. The system of claim 19, wherein the smart pill container further comprises a light associated with each pill compartment, wherein the microcontroller is configured to illuminate the light associated with the pill compartment corresponding to the current day and time when opening of any of the pill compartments is detected.
